(19) United States
(12) Patent Application Publication
(10) Pub. No.: US 2005/0065445 A1
(43) Pub. Date: Mar. 24, 2005

(54) Cardiac arrest monitor and alarm system

(76) Inventors: Robert C. Arzbaecher, Chicago, IL (US); Janice M. Jenkins, Chicago, IL (US); Michael C. Garrett, Wilmette, IL (US)

Correspondence Address: Douglas H. Pauley;Pauley Petersen & Erickson, Suite 365, 2800 West Higgins Road, Hoffman Estates, IL, 60195, US

(21) Appl. No.: 10/979,995

(22) Filed: Nov. 3, 2004

Related U.S. Application Data
(63) Continuation-in-part of application No. 10/437,336, filed on May 13, 2003.
(63) Continuation-in-part of application No. 10/153,458, filed on May 22, 2002.
(60) Provisional application No. 60/292,672, filed on May 22, 2001.

(51) Int. Cl. A61B 5/0402

(52) U.S. Cl. 600/515; 600/509

(57) ABSTRACT

A cardiac arrest monitor and alarm system including an implantable medical device having at least three electrodes, preferably but not necessarily subcutaneous, positioned with respect to a heart organ and forming an orthogonal lead configuration to continuously monitor an electrocardiographic signal of the heart organ. A microdevice, preferably but not necessarily operatively connected to the medical device, detects a deviation from a normal heart electrical activity and emits a signal to an external receiver. Upon verification of the signal from the microdevice, the external receiver activates a programmed annunciator circuit to alert bystanders to deploy an AED and/or activate a communication link automatically transmitting an alarm and the electrocardiographic signal to a remote transceiver.

### CROSS REFERENCE TO RELATED APPLICATIONS

This application is a continuation-in-part of application Ser. No. 10/437,336, filed 13 May 2003, and a continuation-in-part of application Ser. No. 10/153,458, filed 22 May 2002, which claims the benefit of U.S. Provisional Application No. 60/292,672, filed 22 May 2001, the entire disclosures of which are incorporated into this application by reference thereto.

### BACKGROUND OF THE INVENTION

1. Field of the Invention

This invention relates to a cardiac arrest monitor and alarm system that continuously monitors a patient's heart and detects a deviation from a normal heart electrical activity to alert bystanders and activate a communication link to transmit an alarm and an electrocardiographic signal to a remote transceiver, which permits automatic geographical location of the patient.

2. Description of Related Art

Sudden cardiac death, cardiac arrest due to ventricular fibrillation or, in some cases, profound bradycardia and asystole, is the major cause of death in the economically developed world. With over 300,000 cardiac arrests in the United States each year the chances of survival in maj or urban settings and most communities is 3-5%. These people die largely because life-saving external defibrillators arrive on the scene too late. Paramedical personnel use full-featured manual external defibrillators, but the relatively small number of paramedical vehicles in the United States results in responses too late for a reasonable chance of survival. For example, with rapid defibrillation in the Chicago airports survival rates have increased to 65% and similar rates are seen in casinos where rapid defibrillation systems have been implemented. If rapid defibrillation can be accomplished in less than 3 minutes from the time of arrest, survival for these same patients can be over 65%. Data from internal cardiac defibrillators reveal an even higher rate of survival because these devices defibrillate ventricular fibrillation automatically within seconds.

The primary obstacle to better survival is length of time to defibrillation. For every minute of delay, the survival rates decrease by about 10%. Two factors combine to delay rescue. First, in many cities the time for Emergency Medical Service (EMS) or Patient Monitoring Service (PMS) or other similar rescuers to respond to a patient is too long. Consider Chicago and New York where time intervals to defibrillation were about 16 minutes. Public access defibrillation (PAD) programs may help some of these patients to receive faster lifesaving defibrillation in public places, but PAD programs do not work for the majority of patients who suffer an arrest at home or where the collapse is unobserved. Studies show that about 80% to about 85% of cardiac arrests occur in the home, not a public place where rescuers can activate a PAD system. Even more disturbing, nearly 50% of arrest victims are unwitnessed. An unwitnessed cardiac arrest victim has a less than 2% chance of survival. Improving survival of patients who have arrests at home or are unwitnessed will not be improved by therapy devices, such as AEDs and PAD programs, unless a new method for early detection of cardiac arrest is developed.

Cardiac arrest is a persistent clinical problem due to three factors: the inability to predict arrhythmic events; inefficient measurement and maintenance of anti-arrhythmic drug levels in the field; and the evolving metabolic substrate of the myocardium. These factors make the immediate and prolonged application of anti-arrhythmic medicines a complicated task. Aggressive risk stratification of patients has attempted to impact survival but these strategies have been stymied by the transient nature of the acute coronary syndromes. Impacting survival in “out of hospital” cardiac arrests will require alternative approaches that improve public response to resuscitation and measures to prevent unstable coronary lesions.

The substrate in sudden cardiac death is roughly approximated to include about a 50/50 breakdown between patients who have had a remote myocardial infarction (MI) and those who have had their initial ischemic or infarct event. Death from VT/VF occurs in approximately 50% of acute myocardial infarction patients before arriving at a hospital. A 4% to about 18% risk of primary VT in MI exists within the first four hours of plaque rupture. The problem, especially in larger urban cities, has been that the prolonged time to first defibrillation shock has resulted in dismal mortality rates in sufferers of acute coronary syndromes and sudden cardiac death. New paradigms of intervention in the management of out of hospital VT/VF are evolving. Strategies including early response to the detection of symptoms, rapid revascularization, public education into cardiopulmonary resuscitation (CPR) as well as automatic external defibrillation (AED) are improving survival. These strategies are being applied to reduce the incidence of VT and shorten the time to defibrillation, and in turn decrease the extent of anoxic encephalopathy.

Ventricular fibrillation (VF) in acute transmural myocardial infarction (AMI) and the acute coronary syndromes is a sudden arrhythmia that contributes to the majority of sudden cardiac deaths from the earliest onset of symptoms to reperfusion to anytime following formation of a remodeled scar. Three categories of VT are generally accepted: primary VF associated with MI or ischemia in the absence of shock or severe end stage heart failure; primary VT not associated with MI (poor ejection fraction [EF]±coronary disease); and secondary VT which occurs in patients in shock or severe end stage heart failure. Why certain patients have a predilection for VT in Ml or ST segment elevation while others with similar clinical presentation do not, is largely a mystery and likely multifactorial with infarction and ischemia providing a common denominator.

The true incidence of sustained ventricular tachycardia (VT) in acute coronary syndromes is difficult to pinpoint although VF is certainly the most common ultimate rhythm in sudden cardiac death associated with MI. Whether monomorphic VT is the initiating arrhythmia is the subject of considerable debate and speculation. In a combination of AMI and remote MI patients, VT has been reported as the instigating arrhythmic substrate in 62% of the population (n=157). Still, the outcome in hemodynamically compromising VT is certainly as mortal. Reentrant VT is not beyond the pathologic scope of the acutely infarcting myocardium dependent on the timing and amount of tissue damaged. Rapid idioventricular rhythm can also be a consequence of reperfusion and often indicates a positive effect during the infusion of thrombolytics.

Bradytachycardia as a result of MI is not uncommon, particularly in patients with inferior and posterior wall involvement. Mechanisms of bradycardia in acute myocardial infarction can involve the conduction bundles directly or abnormally exaggerated neurocardiogenic reflexes (i.e., Bezold-Jarisch Reflex). Complete heart block or acute bifasicular block during MI implies a more extensive infarct zone and inadequate collateral blood flow and is a poor prognostic sign and may signify a greater predisposition to pump failure.

Technology for monitoring the high-risk cardiac patient was introduced during the last half of the last century, when intensive care units were first established. The technology consisted of bedside ECG monitors permanently connected to the patient and equipped with algorithms for measuring heart rate and the presence of premature ventricular contractions. The devices alarm the staff when a life-threatening arrhythmia is present or frequent ventricular ectopy suggests that such an arrhythmia is imminent.

Outside the hospital, the cardiac patient is monitored with a small recorder (“Holter” recorder) strapped to the patient and connected to several EGG electrodes on the upper torso. In this application there is no alarm; the records are retrieved later to be scanned for occurrences of slow or fast heart rates and ectopic activity. The results are used to guide drug therapy or pacemaker/defibrillator implantation.

An implantable version of the ambulatory recorder is activated by the patient or automatically stores symptomatic EGG episodes. The device is used primarily in the diagnosis of unexplained episodes of fainting, and is implanted after all non-invasive and invasive stratifying tests are negative. The device uses a hermetically sealed can to house the circuitry and battery with electrodes positioned on the ends of the can, giving a single lead for ECG storage. The patient positions a magnet over the can just before, during or just after an event to trigger the ECG storage mechanism and mark the time and date. No alarm is given; the device's memory is downloaded during an office visit and the results are used to guide therapy, as with the Holter monitor. In addition, the electrodes are on the surface of the device and, since the device is designed for implantation, the electrodes are necessarily close together. This close spacing can result in an ECG signal that is of low amplitude and is overly sensitive to the source and direction of electrical activation in the heart. Such a signal would be difficult to process automatically, particularly when the electrical activation changes dramatically, as it does during some dangerous rhythms.

Another monitoring device called the Watchman includes a wrist watch transmitter that can be activated by the patient at the time of symptoms, in order to alert a medical monitoring service. The use of the device requires subscription to a central monitoring service, which serves as an intermediary to summoning rescue. However, the device does not provide for locating the victim.

Although they are intended primarily for the delivery of electrical therapy, Internal Cardioverter Defibrillators (ICDs) also serve as monitors. These devices have the ability to log events of high rate when detection criteria are met. The event logs include intracardiac electrograms from tip to distal right ventricular coil or far field electrograms from RV coil to superior vena cava coil or RV coil to left or right pectoral can. Collection is based on a rolling method with the most recent events replacing older events.

Detection of life-threatening arrhythmias is a function of all implantable cardioverter/defibrillators (ICDs). The algorithms used are complex because false detection of an arrhythmia where none was present results in a very painful shock and may even induce an actual arrhythmia. Yet the ICD must not underdetect either, since the untreated arrhythmia is often fatal.

Further, conventional ICDs do not have the capability of detecting acute ischemia by measurement of ST segment deviation. Yet acute ischemia is frequently a precursor of life-threatening arrhythmia, and many minutes are saved if rescuers can be called at the onset of ischemia, before the actual arrhythmia develops.

Algorithms for detecting VT/VF depend on accurate detection of ventricular activation and measurement of the interval between activations. When all or most such intervals are shorter than a pre-set number, hemodynamically unstable VF or VT is diagnosed. The sensitivity of these devices in high rate detection is 99.8%, while the specificity is approximately 70%. To alter this low specificity, sensing enhancements to discriminate non-life threatening supraventricular tachycardias, such as constancy of rate and suddenness of onset, can be enabled as well.

The detection of ventricular activation in conventional ICDs is greatly simplified by the fact that the sensed ECG is derived from intracardiac electrodes and contains distinct and easily discernable complexes even in a disorganized rhythm such as VT. However, the algorithms do not accurately sense the high rate and erratic rhythm of VT from the rather broad, indistinct, and variable complexes derived from the thoracic subcutaneous surface.

There is an apparent need for a cardiac arrest monitor and alarm system that automatically calls for help and the deployment of a therapy device, such as an automatic external defibrillator (AED), for patients who suffer unwitnessed cardiac arrest.

There is an apparent need for a cardiac arrest monitor and alarm system having an alarm that is integrated with a remote transceiver, for example the EMS or PMS system to reduce the time expired before rescues.

There is an apparent need for a cardiac arrest monitor and alarm system that is integrated with a therapy device, such as an AED.

Further, there is an apparent need for a device that detects or recognizes VT/VF using skin and/or subcutaneous electrodes.

Additionally, since acute myocardial ischemia (AMI) is a prelude to cardiac arrest, there is an apparent need for a device that detects or recognizes a key indicator of AMI, elevation above or depression below a baseline of the segment of the ECG following the QRS complex.

### SUMMARY OF THE INVENTION

It is an object of the present invention to provide an implantable medical device for monitoring a patient's heart rhythm by automatic detection of heart beats from a vector magnitude ECG signal, and to provide an alarm unless normal heart rhythm is detected.

It is another object of the present invention to provide a system for providing an alarm when a deviation from a normal heart electrical activity occurs.

It is yet another object of the present invention to provide a system for providing an alarm when a ST segment deviation indicates the presence of acute ischemia.

The above and other objects of the invention are accomplished with an implanted microdevice that notifies bystanders and/or a remote transceiver, for example an Emergency Medical Service (EMS) or Patient Monitoring Service (PMS) of an incipient cardiac arrest and/or acute myocardial ischemia. Such notification can shorten materially the time to defibrillation of most witnessed, and all unwitnessed, episodes of cardiac arrest, thereby improving survival manyfold. The microdevice will automatically detect the lethal event and signal transcutaneously to an external receiver small enough to be worn on the belt, carried in a purse, worn around the neck as a pendant, worn on the wrist, taped to the skin, or to be positioned or mounted with respect to the user in another similar manner, or to be placed on a local desk or night stand or mounted on the wall. The external receiver gives voice instructions or another similar warning to bystanders to deploy a therapy device, such as an AED and transmits an alarm and the patient's ECG signal to the remote transceiver, such as the nearest EMS or PMS, allowing victim location. Alternatively, for use in the home, the microdevice will signal transcutaneously to at least one of a plurality of external receivers connected to or plugged into existing wall electric supply receptacles preferably, but not necessarily, located in each room of the home. The external receiver detecting the signal will forward the signal to a telephone programmed to automatically call an emergency telephone number, such as the nearest EMS or PMS. Candidates for the implanted device are those readily identifiable cardiac patients whose medical condition and/or history puts them at particularly high risk of cardiac arrest.

Because “false alarms” are easily discounted by the patient and are not a serious problem in the present invention, the entire philosophy of detection can be quite different from that universally used in ICDs and other monitoring devices. This invention does not detect and distinguish among the various arrhythmias (ventricular tachycardia, fibrillation, asystole) and then activate an alarm. Rather, this invention detects the presence of normal rhythm and simply withholds the alarm while normal rhythm is maintained. This provides a higher level of sensitivity and lower level of specificity than existing devices, thus allowing fewer false negatives (underdetecting) and more false positives (overdetecting). Further, the device notifies the patient of an alarm condition before sending the alarm, thereby allowing him or her to disable the alarm if no symptoms are present. The consequence is an algorithm which is simpler in design and implementation.

The cardiac arrest monitor and alarm system includes an implantable medical device having at least three subcutaneous electrodes connected by lead wires to, or located upon a surface of, a small microdevice implanted under a patient's skin and at least one external receiver that can be carried by the patient in a purse or pocket or attached to a belt, or placed on a nearby desk, table, night-stand or wall electric supply receptacle. The implanted microdevice monitors the patient's electrocardiographic signal to detect a deviation from a normal heart electrical activity, and transmits a signal transcutaneously when a life-threatening event is occurring or imminent. The external receiver receives the signal, emits a local alarm, for example to alert any bystanders of an emergency situation and/or to deploy a therapy device, such as an AED, and/or activates a communication link with a remote transceiver to transmit an alarm and the patient's ECG signal to the remote transceiver.

At least three electrodes for obtaining continuous electrocardiographic signals are positioned under the skin, or subcutaneously, and connected to differential inputs of a sense amplifier. Each of the plurality of sense amplifiers receives a electrocardiographic signal from one orthogonal lead and each sense amplifier emits an amplified and digitized ECG signal to a microcontroller for processing, which may be implemented as a logic state machine or microprocessor, for example. The microcontroller executes a stored set of instructions to analyze the ECG signal, determine a deviation from a normal heart electrical activity, for example the onset of a heart attack or lethal heart rhythm, and activate a radio frequency transmitter, for example. The transmitter, when activated, transmits a warning signal, for example to deploy a therapy device, such as an AED, as well as the victim's ECG through the skin to the external receiver.

The radio frequency signal transmitted from the implanted microdevice is received by an antenna located with respect to the external receiver and is fed to a processor within the external receiver. The processor detects the signal and activates a programmed annunciator circuit that delivers a voice message loud enough to be heard by nearby persons, commanding deployment of a therapy device, such as an AED. Additionally, the processor activates a cell phone or, alternatively, a telephone interface circuit which automatically dials 911 and establishes a communication link with a remote transceiver, for example an EMS, PMS or other similar rescuer. In addition to allowing 2-way voice communication between any nearby person and the remote transceiver, such as the EMS or PMS dispatcher, the communication link also transmits an alarm and the patient's ECG signal to the remote transceiver and automatically provides for locating the patient using conventional telephone call tracing, a recently mandated FCC enhanced 911 automatic location identification system or a GPS system, for example.

Other objects and advantages of the present invention will be apparent to those skilled in the art from the following detailed description taken in conjunction with the appended claims and drawings.

### BRIEF DESCRIPTION OF THE DRAWINGS

The drawings show different features of a cardiac arrest monitor and alarm system, according to preferred embodiments of this invention, wherein:

FIG. 1 is a schematic drawing of a patient showing placement of the implantable medical device subcutaneously, having a plurality of electrodes and a microdevice positioned with respect to the patient's heart to form an orthogonal lead configuration, according to one preferred embodiment of this invention;

FIG. 1A is a schematic drawing of a patient showing placement of the implantable medical device subcutaneously, having a microdevice and a plurality of electrodes located on an exterior surface of the microdevice and positioned with respect to the patient's heart to form an orthogonal lead configuration, according to one preferred embodiment of this invention;

FIG. 2 is a schematic block drawing of subcutaneous electrodes operatively connected to a microdevice, according to one preferred embodiment of this invention;

FIG. 2A is a schematic block diagram of a cardiac arrest monitor and alarm system showing a patient with a microdevice in communication with various types of external receivers and/or alarms, according to one embodiment of this invention;

FIG. 3 is a schematic block drawing of an external receiver electromagnetically connectable to an implantable medical device and in communication with a remote transceiver and/or a therapy device, such as an AED, according to one preferred embodiment of this invention;

FIG. 4 is a flowchart diagram of an algorithm used in processing an electrocardiographic signal of a patient's heart to detect a deviation from a normal heart rhythm, according to one preferred embodiment of this invention; and

FIG. 5 is a flowchart diagram of an algorithm used in processing an electrocardiographic signal of a patient's heart to detect a presence of a ST elevation or depression, characteristic of acute ischemia.

### DETAILED DESCRIPTION OF PREFERRED EMBODIMENTS

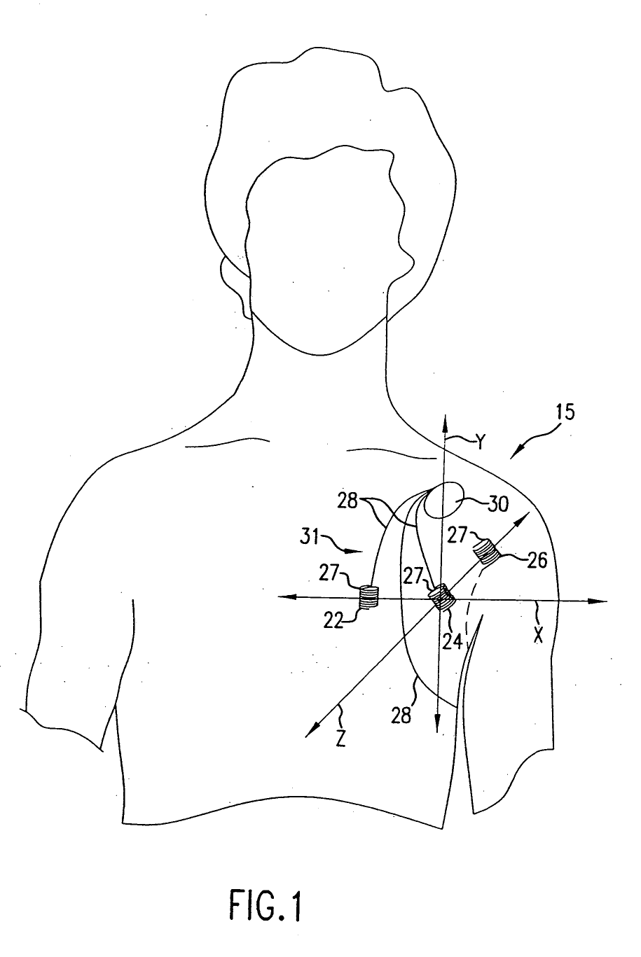

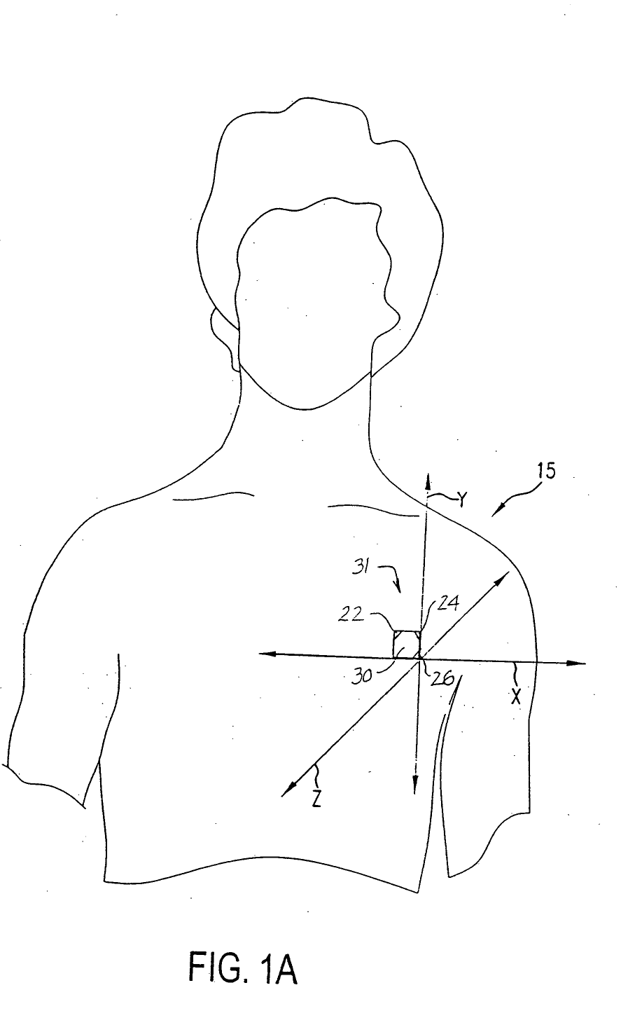

As shown in FIGS. 1-3, in one preferred embodiment of the invention, a cardiac arrest monitor and alarm system comprises an implantable or subcutaneous medical device 15. In one preferred embodiment of this invention, medical device 15 is chronically and completely implanted within a patient's body. Medical device 15 can be implanted subcutaneously to sample electrocardiographic (“ECG”) signals to continuously monitor and analyze the ECG signals to detect normal cardiac electrical activity and automatically initiate communication with an external communicating device to warn a bystander to deploy a therapy device, such as an AED when the results of the analysis call for medical intervention. Throughout this specification and the claims, the terms therapy device and AED are intended to be interchangeable with each other and are intended to include any known or future method and/or apparatus for applying therapy to a heart organ or other monitored organ.

In one preferred embodiment of this invention, medical device 15 comprises at least three subcutaneous electrodes 22, 24, 26 positioned with respect to a heart organ, for example a human patient's heart to form a subcutaneous orthogonal lead configuration or system 31 to continuously monitor the ECG signals of the patient's heart. In order to obtain an ECG signal of high quality, suitable for processing in order to generate an alarm automatically, subcutaneous electrodes 22, 24, 26 are preferably spaced at least 4.0 centimeters apart from each other and positioned in the precordial region of the chest of the patient. Lead wires 28 are tunneled under the skin between electrode 22, 24, 26 and an implanted microdevice 30. Further, electrodes 22,24,26 are positioned at the corners of a 2-dimensional or, preferably, 3-dimensional parallelepiped to group electrodes 22, 24, 26 in pairs such that each pair forms with other pairs a set of orthogonal electrocardiographic leads.

Representation of the electrical nature of the heart as a current dipole is a well known concept in electrocardiography. In this concept, the heart is modeled as a single source of current and a sink for that current, wherein the source and sink are closely spaced points within the chest in the region occupied by the heart. It is apparent to those skilled in the art that a current dipole immersed in a conducting medium such as the body will create voltages throughout the body and upon its surface that are ideally measured by three pairs of electrodes whose axes, the straight lines joining the two electrodes of each pair, are mutually perpendicular to each other. This is the basis of that branch of electrocardiolography called “vectorcardiography.” The three leads, such as connections of electrodes, used in vectorcardiography are called “orthogonal leads” because they measure the three perpendicular components of the cardiac dipolar source: vertical; horizontal anteroposterior; and horizontal transverse. In principle, from these three components the cardiac source can be accurately reconstructed. All the information needed to describe the heart's dipolar electrical activity is contained in these three leads. A reduced set of two such leads can describe the heart's electrical activity in two dimensions, for example, in a frontal plane. Depending on the direction of electrical activation across the heart, the three components of the cardiac dipole will vary in relative magnitude, but the composite or vector magnitude will not. In particular, the vector magnitude of the three signals from the orthogonal lead set will, during a dangerous rhythm, be similar to that obtained during normal rhythm, since the vector magnitude is independent of the source and direction of cardiac activation.

As used herein, the term “orthogonal lead” refers to an electrocardiographic connection between two or more electrodes and the term “orthogonal lead configuration” refers to a set or plurality of orthogonal leads which form right angles with each other or are mutually perpendicular to each other. In one preferred embodiment of this invention, the orthogonal lead configuration 31 is three dimensional (FIG. 1). However, in certain embodiments, the orthogonal lead configuration 31 may be two-dimensional, for example in the frontal plane (FIG. 1A).

Medical device 15 further comprises implantable microdevice 30 operatively connected to each electrode 22, 24, 26 for continuously monitoring a normal heart electrical activity and detecting a deviation from the normal heart electrical activity. The term “deviation” refers to rhythm abnormalities, including ventricular fibrillation (“VF”) and ventricular tachycardia (“VT”), as well as repolarization changes in the ECG signal that may be related to ischemia, such as an elevation or a depression of a ST-segment. The presence of ischemia correlates positively with a high risk for the development of ventricular fibrillation or other forms of sudden cardiac death. The frequency and duration of active ischemia characterizes the severity of the risk.

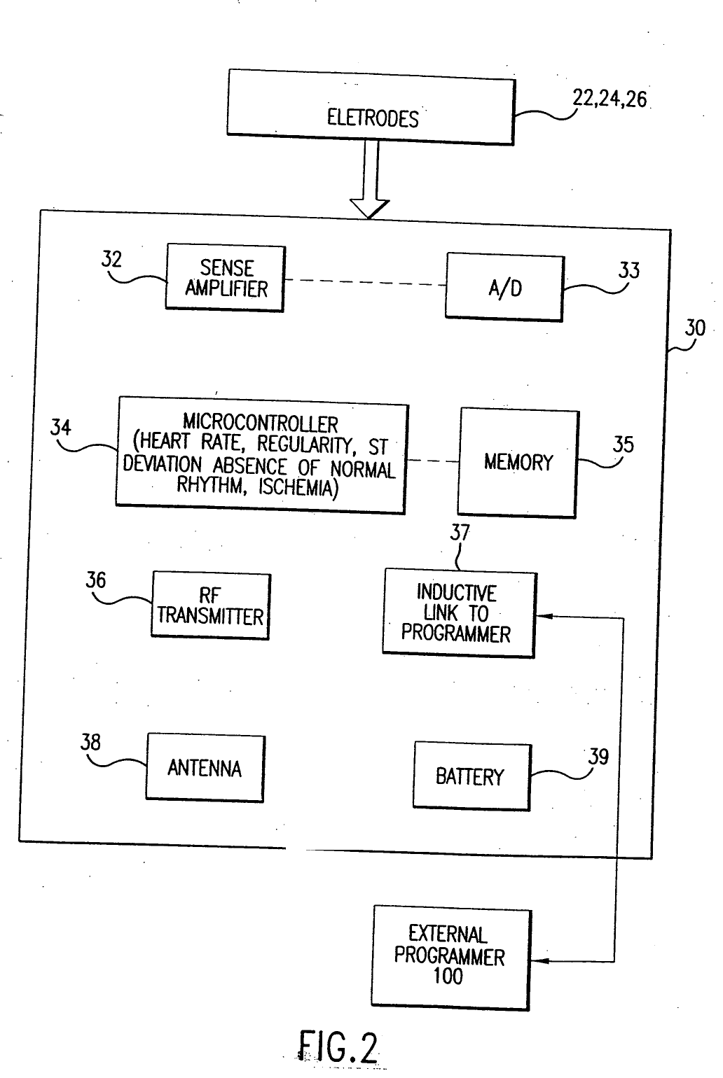

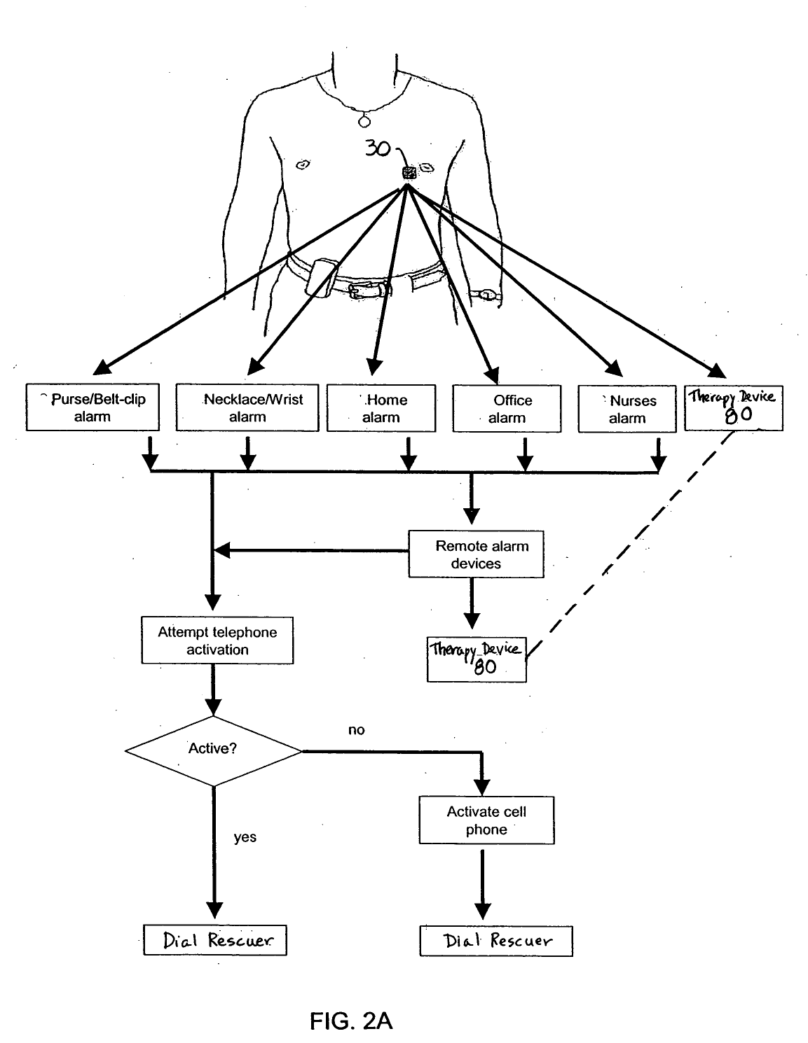

Preferably, microdevice 30 comprises a biocompatible metallic enclosure or casing, for example a titanium enclosure. It is apparent that microdevice 30 may be made of any suitable biocompatible metallic enclosure known to those having ordinary skill in the art. Microdevice 30 emits a signal to an external receiver 60, which detects the emitted signal and activates a programmed annunciator circuit 62 to alert bystanders, for example to deploy a therapy device, for example an AED 80 such as shown in FIG. 2A, and activates a communication link 66 automatically transmitting alarm 47 and the ECG signal to a remote transceiver 90, which allows or permits the automatic location of cardiac arrest monitor and alarm system 10. The therapy device can contain an alarm responsive to external receiver 60 and/or microdevice 30.

In one preferred embodiment of this invention, implanted medical device 15 comprises subcutaneous precordial implantation of electrodes. Electrodes 22, 24, 26 are implanted subcutaneously in the fatty tissue beneath the dermis but above the muscle fascia. Implantation in this manner involves minimal surgical invasion and no invasion of the heart.

Each electrode 22,24,26 preferably comprises a conducting helical coil 27 having a length about 1.0 centimeter (cm) to about 2.0 cm and a diameter about 2.0 millimeters (mm) to about 4.0 mm. Conducting helical coil 27 provides for maximum fatigue resistance. It is apparent that electrodes 22, 24, 26 may have any suitable shape and/or dimensions. Preferably, each electrode 22, 24, 26 is located or positioned at a distal end of one insulated lead wire 28. A proximal end of each lead wire 28 is electrically connected to microdevice 30. Any suitable electrical connection may be used to connect each electrode 22, 24, 26 to microdevice 30.

Electrodes 22, 24, 26 sense cardiac signals and wires 28 conduct the electrical signals from electrode 22, 24, 26 to microdevice 30. Implanted wire 28 must be corrosion free and biocompatible, must reliably carry signals for a number of years, resist dislocation over time and withstand conditions of external or internal stresses. Wire 28 is implanted within the subcutaneous layer of tissue and is subject to greater mechanical strains than are cardiac pacemaker leads for example which, other than at the ends of the lead, can move more freely within a blood vessel. As compared to cardiac pacemaker leads, wire 28 should have additional strength and a superior ability to withstand mechanical stresses due to flexing, torsion, and elongation. At points along wire 28 other than at the locations of electrodes 22, 24, 26, the conductor is isolated from the body using polyurethane or Silastic insulation. In one preferred embodiment of this invention, wire 28 comprises a polyurethane insulation to provide toughness and higher tensile strength.

In one preferred embodiment of this invention, electrodes 22,24,26 and microdevice 30 form orthogonal electrocardiographic lead system 31, as shown in FIG. 1A. Electrode 22 is positioned adjacent and left of the sternum at the third intercostal space; electrode 24 is positioned at a horizontal level of electrode 22 and positioned about halfway between the sternum and the left midaxillary line; microdevice 30 or the enclosure of implantable medical device 15 is about 6.0 cm above electrode 24; and electrode 26 is on a patient's back directly posterior to electrode 24. It can be seen that these electrodes 22,24,26 and microdevice 30 are approximately positioned at four corners of a rectangular parallelopiped. During implantation, subcutaneous placement of electrodes 22, 24, 26 and microdevice 30 is selected to optimize ECG amplitude during sinus rhythm and VT/VF, and maximize observation of significant ST elevation/depression during AMI. Electrocardiographic voltages obtained between electrode 24 and electrode 22, electrode 24 and microdevice 30, electrode 24 and electrode 26 define three orthogonal lead signals and measure the X, Y and Z components, respectively, of the cardiac dipole.

In one preferred embodiment of this invention as shown in FIG. 1A, medical device 15 comprises a plurality of subcutaneous electrodes, for example three subcutaneous electrodes 22, 24, 26 preferably located or positioned on an outer or exterior surface of implanted microdevice 30 and at the corners of a 2-dimensional parallelopiped to group electrodes 22, 24, 26 in pairs such that each pair forms with other pairs a set of orthogonal electrocardiographic leads. Each electrode 22, 24, 26 is electrically connected to microdevice 30 by lead wire 28 that is preferably contained entirely within microdevice 30. Preferably, each electrode 22, 24, 26 is located or positioned at a distal end of insulated lead wire 28. A proximal end of lead wire 28 is electrically connected to microdevice 30. Any suitable electrical connection may be used to connect each electrode 22, 24, 26 to microdevice 30.

Referring to FIG. 1A, in one preferred embodiment of this invention, electrodes 22, 24, 26 and microdevice 30 form orthogonal electrocardiographic lead system 31. In this preferred embodiment, the simpler configuration of electrodes 22, 24, 26 with respect to microdevice 30 results in closer spacing of electrodes 22, 24, 26 compared to one preferred embodiment as shown in FIG. 1 for example, but two-dimensional orthogonality is preserved so that the vector magnitude of the ECG signal is not overly sensitive to the source and direction of cardiac activation.

In one preferred embodiment of this invention, implantable medical device 15 comprises a number of components. For example, implantable medical device 15 may include at least one suitable component as described in U.S. Pat. No. 5,113,869 issued to Nappholz et al. on 19 May 1992, the disclosure of which is incorporated herein by reference. In a particular application, some of the components may not be clinically necessary and are optional. Referring to FIG. 2, system components within microdevice 30 include a plurality of sense amplifiers 32, an analog to digital (“A/D”) converter 33, a microcontroller 34, memory 35, a radio frequency transmitter 36, an inductive link 37 to an external programmer 100 and a battery 39. Further, a preferred signal path is shown in FIG. 2. It is apparent to those skilled in the art that in certain embodiments of this invention, the signal path may vary from the path shown. Medical device 15 may further include an antenna 38 mounted with respect to microdevice 30, for example mounted within microdevice 30 or mounted on an exterior surface of microdevice 30.

Referring further to FIG. 2, microdevice 30 comprises a plurality of sense amplifiers 32. In one preferred embodiment of this invention, each orthogonal electrode pair defining an orthogonal lead signal is electrically connected to a differential input of one sense amplifier 32. Preferably, each sense amplifier 32 is a sampling and digitizing amplifier, as is well known to those having ordinary skill in the art. Preferably, but not necessarily, sense amplifiers 32 are characterized by having a high input impedance, a high output impedance, a high common mode rejection, a sensitivity of about 8 to about 10 bits per millivolt, and a sampling rate of about 250 Hz. Further, each sense amplifier 32 contains a digital filter having a passband of about 0.05 Hz to about 500 Hz.

As shown in FIG. 2, microdevice 30 further comprises a microcontroller 34 electrically connected to each sense amplifier 32. Microcontroller 34 receives the amplified, digitized and filtered ECG signals and further processes the ECG signals to detect a deviation from the patient's normal heart electrical activity. Each sense amplifier 32 receives an ECG signal from an orthogonal lead and each sense amplifier 32 emits an amplified and digitized ECG signal to microcontroller 34 through A/D converter 33, each positioned within microdevice 30.

Microcontroller 34 controls the other components of cardiac arrest monitor and alarm system 10. In particular, microcontroller 34 controls transmitter function 36, memory reading and writing 35, acquisition of sensed signals 32, and real-time clock functions to provide the capability of shutting down the entire system when idle. Microcontroller 34 provides standard functionality but also includes a boot ROM, timers, a watchdog timer and an input/output (i/o) port. The watchdog timer is an emergency circuit providing a power-up reset if microcontroller 34 remains idle for longer than a preset period. The i/o port allows communication between microcontroller 34 and other circuit elements with no need for extra circuitry outside microcontroller 34. The boot ROM configures software in RAM memory to a ready state for power-up operations upon system reset. The fast clock is a high frequency oscillator, for example, 3 MHz, which drives the instruction timing within microcontroller 34.

Within microcontroller 34 there are maskable wakeup circuitry in the form of a data register which enables and disables the ability of microcontroller 34 to detect wakeup signals from sense amplifiers 32 and the real-time clock. Timers within microcontroller 34, transmitter 36, sense amplifiers 32 and the real-time clock generate signals which signify pertinent events. Microcontroller 34 determines when and how to respond to these events by means of mask registers which selectively allow microcontroller 34 to ignore or respond to such events.

Transmitter 36 transfers data and programs between implanted microdevice 30 and an external receiver 60. While performing normal operations, implanted microdevice 30 will receive from external programmer 100 downloaded program object code and other control information to govern data acquisition by microdevice 30. Under the direction of commands from external programmer 100, microdevice 30 will reply with acquired and processed physiological data. It also is a normal operation for microdevice 30 to acquire and process the physiological data and analyze the data to detect warning conditions. In this mode of operation, determined by and under the direction of the downloaded program code, microdevice 30 may initiate communication with external receiver 60 to warn of abnormal physiological conditions, such as those that warrant the deployment of a therapy device, such as an AED. Microdevice 30 may also warn external receiver 60 of a malfunction within implanted medical device 15 in response to an attempt and failure of a self-diagnostic test. Transmitter 36 is preferably a radio frequency transmitter which directs data flow from the rf circuits to the-data bus under the control of microcontroller 34. The implantable microdevice 30 transmits information to external receiver 60 with a range of at least 20 feet at a telemetric data transmission frequency preferably of about 400 MHz.

In one preferred embodiment of this invention, cardiac arrest monitor and alarm system 10 comprises a plurality of external receivers 60 electromagnetically connected to microdevice 30. For example, one external receiver 60 may be positioned within each room of a house and connected to the house power line via an electric supply receptacle located in each room. Each receiver 60 is capable of detecting a signal transmitted transcutaneously from microdevice 30 alerting a bystander to employ a therapy device, such as an AED, and/or forwarding or transmitting the detected signal to a telephone, which can be programmed to automatically dial a desired emergency telephone number, such as the nearest EMS, PMS or other similar rescuer. Each external receiver 60 can-transmit or forward the signal to the telephone using a short-wave radio transmission or a modulated carrier on the household power line, for example. The external receiver may also transmit a signal to activate a therapy device, such as an AED, or to activate more remote alarm devices thereby extending the range at which an alarm can be effective.

Under the control of microcontroller 34, implantable medical device 15 senses ECG signals from sense leads 28 which are in electrical contact with electrodes 22, 24, 26. The ECG signals on sense leads 28 first proceed to sense amplifiers 32 for initial filtering and processing. Sense amplifiers 32 include a standard true instrumentation amplifier with a programmable bandwidth, allowing microcontroller 34 to tailor the signal filtering parameters to a particular type of ECG features as may be required to perform a desired task. A true instrumentation amplifier characteristically has a high input impedance, full differential amplifiers on the input and output, high gain, and an adjustable input resistance. For example, bandwidth requirements vary when performing diverse operations such as measuring ST-segment changes, monitoring heart rate and acquiring high quality ECG signals. The true instrumentation amplifier produces the high quality signal necessary for detailed ECG analysis by filtering input noise and providing minimal phase and baseline shift and a flat amplitude versus frequency response in the selected bandwidth. The wide range of bandwidths allows flexibility in the selection of analysis methods. The programmable bandwidth of sense amplifiers 32 for reproducing high quality ECG signals ranges from the heart rate frequency (commonly 0.05 to 100 Hz). When microcontroller 34 sets the bandwidth in preparation for ST-segment analysis, it selects a much lower frequency range (0.05 Hz to 30 Hz).

Data from sense amplifiers 32 is converted from analog to digital form by analog to digital converter 33. Sense amplifier 32 circuitry includes an anti-aliasing filter to minimize the artifact caused by digitization of the signal. Programming within microcontroller 34 controls the conversion rate within the range from 64 to 1000 samples per second. Programming of microcontroller 34 also directs the digital output of sense amplifier 32 to one or more destinations, for example to transmitter 36 to allow transmission of raw data, to memory 35, or to microcontroller 34 itself for data storage and analysis. Data acquisition control by microcontroller 34 allows the implanted microdevice 30 to constantly analyze data and, in response to that analysis, to perform intelligent data acquisition. For example, microdevice 30 may increase the sample rate or amplifier bandwidth or begin sampling after a pause in response to a particular sensed event.

As shown in FIG. 2, in one preferred embodiment of this invention, microdevice 30 comprises an inductive link 37 to an external programmer 100, such as a monitoring system. External programmer 100 is similar to programmers for interacting with cardiac pacemakers. External programmer 100 may be essentially a computer system with added functionality provided by a telemetry interface wand. The wand includes electromagnetic transmission and reception circuitry similar to that in microdevice 30. The telemetry interface wand receives the signals sent by microdevice 30. Software in external programmer 100 is configured to provide a human interface for controlling the operations performed by microdevice 30. In response to commands of the operator, external programmer 100 reads and displays data from microdevice 30, transmits control parameters to microdevice 30 and downloads diagnostic and application routine machine code from a program library into the RAM memory of microdevice 30. Further, microcontroller 34 is operatively connected to subcutaneous antenna 38 mounted with respect to microdevice 30. Subcutaneous antenna 38 may be mounted within microdevice 30 or may be mounted on an exterior surface of microdevice 30, for example.

The above discussion indicates how the implanted nature of microdevice 30 and the implantation procedure maximize ECG signal quality. Another factor assuring signal quality is the absence of cumulative distortion between the various system components. In one preferred embodiment of this invention, all stored and transmitted signals and information are converted to digital form early in the signal path and maintained in digital form to assure signal quality. No phase distortion is introduced as is the case in external ECG systems. The high quality instrumentation amplifier in the signal acquisition circuitry of implanted microdevice 30 has its bandwidth programmed to produce the optimum signal according to the ECG parameter currently of interest. The signal from the instrumentation amplifier is then digitized for analysis and storage.

Microcontroller 34 processes the ECG signals using at least one algorithm that analyzes the continuously monitored heart electrical activity to detect the deviation from the normal heart electrical activity.

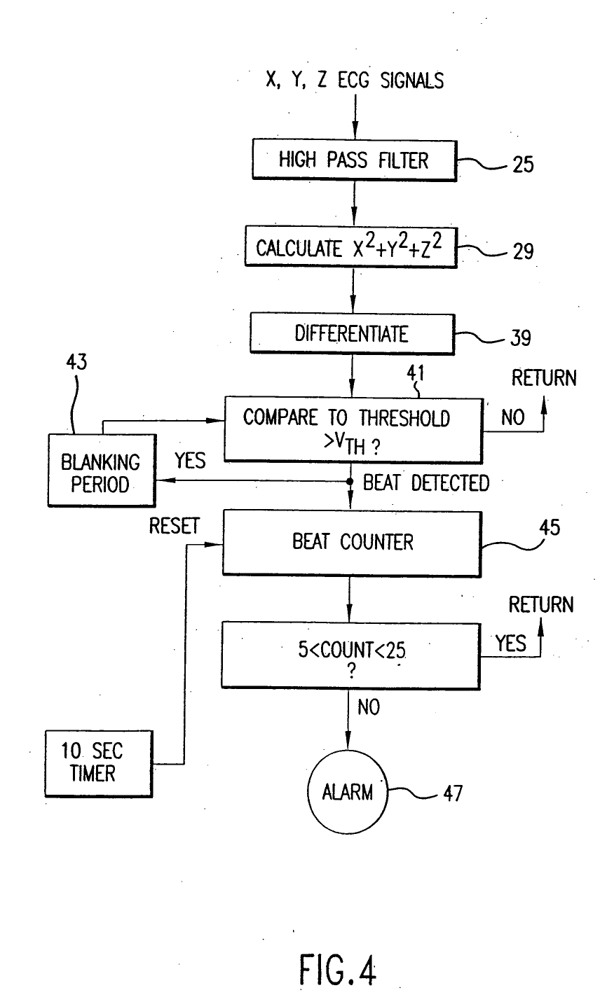

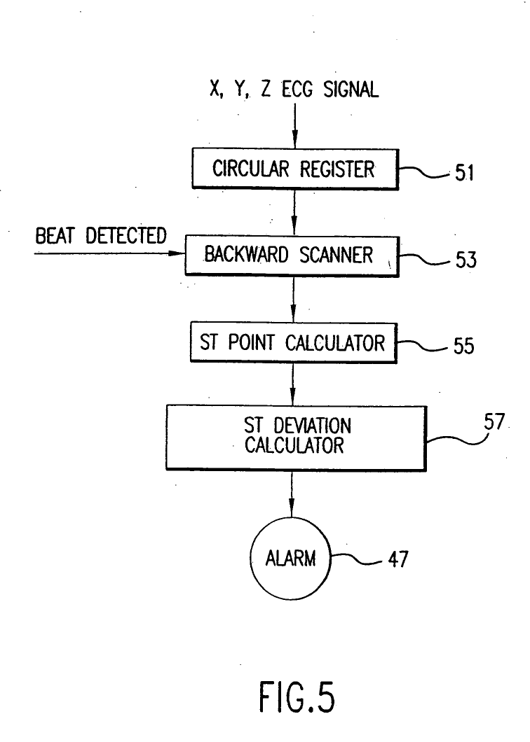

Referring to FIGS. 4 and 5, in one preferred embodiment of this invention, two algorithms for further processing the ECG signals within microdevice 30 can be used to detect a deviation from the normal heart electrical activity. For example, referring further to FIG. 1, the amplified, digitized and filtered ECG signals from the three orthogonal leads are processed and analyzed by microcontroller 34 using an algorithm for detecting any dangerous departure or deviation from the normal heart rhythm, which may indicate cardiac arrest for example. Further, microcontroller 34 measures a deviation from a baseline of a ST segment in each of the three orthogonal leads in order to detect acute ischemia. Alternatively or in addition to microcontroller 34, the processing of the three orthogonal signals can be implemented in hardwired digital circuits.

As shown in FIG. 4, a digital highpass filter 25 with a cutoff frequency of about 5 Hz, for example, receives the three orthogonal lead signals. Digital highpass filter 25 provides baseline stabilization. The digitized ECG signals are processed using a squaring and summing process 29 that produces a vector magnitude signal (V) from the three orthogonal leads X, Y, and Z, according to Equation 1:
X²+Y²+Z²=V Equation 1
The vector magnitude signal is used to detect heart beats and analyze heart rhythm. The vector magnitude signal is led to a differentiator 39 that calculates the spatial velocity vector, which is the derivative of the vector magnitude. Differentiator 39 is followed by a beat detector 41 that identifies each heartbeat by noting when the spatial velocity magnitude exceeds a threshold. Preferably, beat detector 41 is adaptive; it tracks the peak amplitude of the spatial velocity signal by resetting after each detected beat to about 75% of that beat's peak signal, for example, and then tapers off exponentially with a time constant of about 2 seconds, for example. This allows beat detector 41 to adjust the threshold when a sudden decrease in amplitude occurs, for example at an onset of ventricular fibrillation. Following a beat detection there is a blanking (eye-closing) period 43 to prevent multiple recognitions of the same beat. Preferably, blanking period 43 is set at a refractory period of about 150 ms. A beat counter 45 reads and resets every ten seconds to register the number of beats in each ten-second period. Finally, an alarm 47 is generated every 10 seconds unless the number of beats in the ten-second window is within normal limits, for example 5 beats to 25 beats. It is apparent to those having ordinary skill in the art that the numerical values of the parameters given herein are exemplary and other suitable values may be programmed in order to customize the algorithm for a particular patient.

Referring to FIG. 5, a segment of each ECG signal between the end of depolarization (S-wave) and the beginning of repolarization (T-wave) is examined relative to a baseline, in order to determine the presence of a “ST elevation/depression” that is characteristic of acute ischemia. The algorithm uses a circular register 51 to store a plurality of ECG data points immediately previous to a beat detection, for instance the previous fifty ECG data points. Upon detection of each beat, these immediately preceding ECG data points are subjected to a backward scanner 53 to determine the ECG baseline in the PQ segment. The backward scanner 53 works by searching until a passage of several consecutive points are found to be in agreement with a rule of flatness (minimal derivative). The number of consecutive points to be detected will depend upon the sampling frequency. Then a particular data point is determined by a ST Point Calculator 55, which operates in the section of the ECG signal following beat detection, by using the following formula that corrects for heart rate:
ST point=R+n ms+max(4,(200−HR)/16)×4 ms Equation 2
where R denotes the peak of the R wave; HR denotes heart rate computed from the current RR interval; and n designates an interval to be determined from clinical studies.

The elevation of the segment over the baseline or the depression of the segment under the baseline is measured by a ST Deviation Calculator 57. Preferably, but not necessarily, a ST shift of about 0.3 mV or greater activates the alarm 47. It is apparent to those having ordinary skill in the art that all of the numerical values of the parameters given herein are exemplary and other values may be programmed in order to customize the algorithm for a particular patient. Further, similar processes can be performed using the two-dimensional orthogonal leads, such as shown in FIG. 1A, to obtain a vector magnitude signal for detecting heart beats and/or analyzing heart rhythms.

In one preferred embodiment of this invention, subcutaneous transmitter 36 is activatable upon detection of the deviation from the normal heart electrical activity to transmit the alarm 47 and the patient's ECG signal to at least one external receiver 60. External receiver 60 is electromagnetically connected to microdevice 30. For example, external receiver 60 may comprise a cellular phone unit which can be mounted transcutaneously with respect to the patient, such as by using a belt clip or being placed in a pocket or purse. Alternatively, external receiver 60 may comprise a stationary household telephone. In one preferred embodiment of this invention, a receiver 61 within external receiver 60 detects a signal emitted from microdevice 30. Upon receiving the signal from transmitter 36, external receiver 60 actuates a programmed annunciator circuit or voice chip 62 to alert bystanders, for example to deploy a therapy device, such as an AED, and/or activates a communication link 66 with a remote transceiver 90 and/or a therapy device, such as an AED. The bystander alerted by annunciator circuit 62 can communicate with remote transceiver 90 using an integrated cellular phone 75. Communication link 66 automatically transmits alarm 47 and the patient's ECG signal to remote transceiver 90, which permits automatic location of cardiac arrest monitor and alarm system 10. Communication link 66 may comprise a conventional cellular phone or a hardwired household telephone.

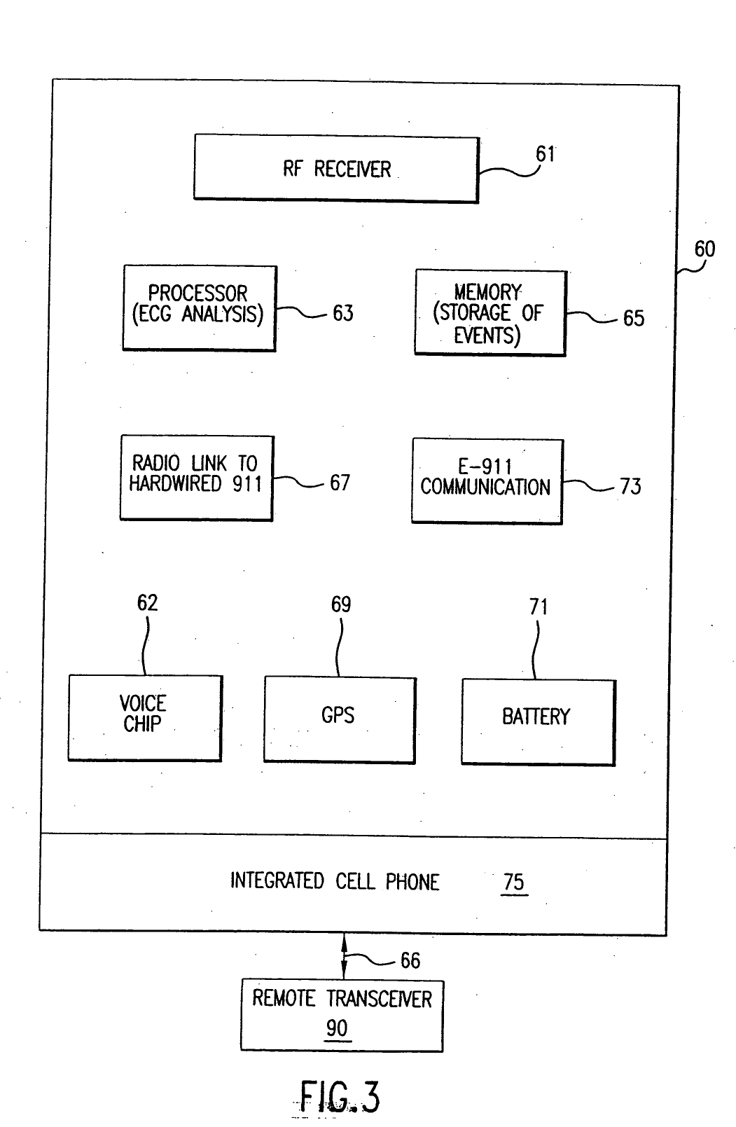

In one preferred embodiment of this invention, external receiver 60 comprises a number of components. In a particular application, some of the components may not be clinically necessary and are optional. Referring to FIG. 3, system components within external receiver 60 include receiver 61 electromagnetically connected to microdevice 30, a processor 63 for further processing and analyzing ECG signals to verify that alarm 47 emitted from microdevice 30 is an accurate detection of a deviation from normal heart electrical activity, and a memory 65 electrically connected to processor 63 for storage of previous ECG events or episodes, a radio frequency link 67 hardwired to a household telephone, for example, which automatically dials an emergency telephone number and/or activates a therapy device, such as an AED, upon detection and verification of alarm 47, a GPS 69, a battery 71, an enhanced 911 identification location signal 73 and integrated cellular telephone 75. Further, a preferred signal path is shown in FIG. 3. It is apparent to those skilled in the art that in certain embodiments of this invention, the signal path may vary from the path shown. External receiver 60 preferably includes an antenna 38 mounted with respect to external receiver 60, for example as an antenna is mounted to a conventional cellular phone.

Preferably, cardiac arrest monitor and alarm system 10 includes all of these capabilities embodied within external receiver 60 that is small and light enough to be attached to the patient when the patient is mobile or to be used by the patient as a free standing unit at the patient's residence or hospital room. Alternatively, cardiac arrest monitor and alarm system 10 can be re-configured in part as a stand alone, line powered, room monitor and the remaining part can be implemented as a patient-worn, battery powered, communications link with a transceiver capable of two-way communication between the patient, the implanted medical device and the line powered monitor.

In one preferred embodiment of this invention, remote transceiver 90 comprises an EMS, PMS or other similar rescuer, and communication link 66 automatically transmits alarm 47 and the patient's ECG signal to the EMS or PMS, which allows the EMS or PMS to locate the patient using enhanced 911 automatic location identification signal 73.

In one preferred embodiment of this invention, external receiver 60 automatically activates a therapy device, such as an AED 80.

In one preferred embodiment of this invention, external receiver 60 comprises Global Positioning System (“GPS”) 69 for determining the geographic location of cardiac arrest monitor and alarm system 10, for example as described in U.S. Pat. No. 6,292,698 issued to Duffin et al. on 18 Sep. 2001, the disclosure of which is incorporated herein by reference. GPS 69 is intended to function no matter how geographically remote the patient may be relative to remote transceiver 90, which may be a monitoring site or medical support network. GPS 69 is activated when alarm 47 is sent from external receiver 60 to remote transceiver 90. Alarm 47 notifies remote transceiver 90 that a system 10 and/or patient problem has occurred, which allows the patient location to be determined via GPS 69. Further, communication link 66 allows a person, for example, the patient or a bystander, to verbally communicate with a monitoring personnel via integrated cellular telephone system link 75. Alternatively, verbal communication may be accomplished using a satellite-based telecommunications link if the patient is outside the range of a cellular link or subscribes only to the satellite-based link.

GPS 69 receives patient positioning data from an earth satellite (not shown). The GPS 69 preferably uses current systems such as the Mobile GPS™ (PCMCIA GPS Sensor) provided by Trimble Navigation, Inc. of Sunnyvale, Calif. or Retki GPS Land Navigation System provided by Liikkura Systems International, Inc. of Cameron Park, Calif., or other similar systems. The GPS 69 may be actuated by a command received by external receiver 60 from the medical support network, in the case of an emergency response. In the case of a non-emergency, periodic follow-up, GPS 69 may be enabled once an hour or once a day or any other chosen interval to verify patient location. It is apparent to those skilled in the art that other suitable locating and data telemetry systems may be included in external receiver 60, for example the systems described in U.S. Pat. No. 5,752,976 issued to Duffin et al. on 19 May 1998, the disclosure of which is incorporated herein by reference, and current or further developed technology.

Referring to FIGS. 1-5, cardiac arrest monitor and alarm system 10 detects a deviation from the normal heart electrical activity and transcutaneously transmits an alarm upon detecting the deviation. The implantable medical device 15 comprises at least three electrodes 22, 24, 26 and microdevice 30 to continuously monitor the ECG signal of the patient's heart organ. Electrodes 22, 24, 26 are positioned with respect to the patient's heart in an orthogonal lead configuration to continuously monitor the ECG signal of the patient's heart organ. The orthogonal lead configuration comprises a set or plurality of orthogonal leads which are generally positioned at right angles with each other.

Each of a plurality of sense amplifiers 32 positioned within microdevice 30 receives a continuous ECG signal from one orthogonal lead. Sense amplifier 32 amplifies and digitizes the ECG signal and feeds the ECG signal to microcontroller 34 positioned within microdevice 30. Microcontroller 34 executes a set of instructions to analyze the ECG signal in order to detect any deviation from the normal heart electrical activity. In one preferred embodiment of this invention, the analysis of the ECG signal comprises operating an algorithm for detecting ventricular activation and measuring an interval between successive ventricular activations. Preferably, the ECG signal analysis further comprises an algorithm for detecting a deviation from a baseline of a segment of the ECG signal following a QRS complex.

Upon detecting the deviation, which may signal an onset of an acute myocardial ischemia, a ventricular tachycardia or a ventricular fibrillation, for example, microcontroller 34 transmits an alarm and the ECG signal to external receiver 60 electromagnetically connected to microdevice 30.

External receiver 60 further processes the ECG signal to verify that a deviation from the normal heart electrical activity is occurring and activates programmed annunciator circuit 62 to emit a local alarm to alert bystanders of a potential life-threatening episode, for example to deploy a therapy device, such as AED 80, and establish communication link 66 with remote transceiver 90 and/or a therapy device, such as AED 80. In one preferred embodiment of this invention, communication link 66 is established by activating a cellular telephone interface circuit 75 to automatically dial a programmed emergency telephone number. Alternatively, communication link 66 may be established by transmitting a radio frequency signal to a hardwired telephone to automatically dial a programmed emergency telephone number. Communication link 66 provides for communication between the patient or a person alerted by annunciator circuit 62 and remote transceiver 90.

Upon establishment of communication link 66, remote transceiver 90, such as an EMS or PMS dispatcher can instruct the person alerted by annunciator circuit 62 to perform life-saving procedures, such as CPR and/or deployment of a therapy device, such as AED 80. Communication link 66 further transmits the alarm and the ECG signal to the remote transceiver 90, wherein a position identifier allows for identification of the patient's location. For example, the position identifier may comprise an enhanced 911 automatic location identification signal or other suitable location signal. In one preferred embodiment of this invention, the transmission of the alarm to remote transceiver 90 can be delayed in order to allow the patient time (several seconds) to disengage the alarm in a false detection of the deviation from the normal heart electrical activity.

While in the foregoing specification the invention has been described in relation to certain preferred embodiments, and many details are set forth for purpose of illustration, it will be apparent to those skilled in the art that the invention is susceptible to additional embodiments and that certain of the details described in the specification and in the claims can be varied considerably without departing from the basic principles of the invention.

1. A cardiac arrest monitor and alarm system comprising:
a medical device having at least three electrodes positioned with respect to a heart organ and forming an orthogonal lead configuration to monitor an electrocardiographic signal of the heart organ;
a microdevice operatively connected to the medical device, each of the at least three electrodes positioned on an exterior surface of the microdevice, the microdevice detecting a deviation from a normal heart electrical activity; and
a plurality of external receivers, at least one of the external receivers detecting a signal from the microdevice and activating a programmed annunciator circuit to activate a local alarm, and activate a communication link automatically transmitting an alarm and the electrocardiographic signal to a remote transceiver.

2. The cardiac arrest monitor and alarm system of claim 1, wherein the medical device is subcutaneous.

3. The cardiac arrest monitor and alarm system of claim 1, wherein the medical device is on a body surface of a patient.

4. The cardiac arrest monitor and alarm system of claim 1, wherein the microdevice is implantable.

5. The cardiac arrest monitor and alarm system of claim 1, wherein the medical device communicates with a position identifier that provides a position of a patient.

6. The cardiac arrest monitor and alarm system of claim 1 wherein the deviation from the normal heart electrical activity is detected with an algorithm that monitors the normal heart electrical activity.

7. The cardiac arrest monitor and alarm system of claim 1 wherein the microdevice comprises a subcutaneous transmitter, and the subcutaneous transmitter is activatable upon detection of the deviation from the normal heart electrical activity to transmit a warning signal and the electrocardiographic signal to at least one external receiver.

8. The cardiac arrest monitor and alarm system of claim 1 wherein the microdevice comprises a plurality of sense amplifiers each receiving an electrocardiographic signal and each emitting an amplified and digitized electrocardiographic signal to a microcontroller within the microdevice.

9. The cardiac arrest monitor and alarm system of claim 8 further comprising an antenna mounted with respect to the microdevice and operatively connected to the microcontroller.

10. The cardiac arrest monitor and alarm system of claim 1 wherein the remote transceiver comprises a telephone, and the communication link automatically transmits the alarm and the electrocardiographic signal to the telephone programmed to automatically call at least one of an Emergency Medical Service, a Patient Monitoring Service and a rescuer.

11. The cardiac arrest monitor and alarm system of claim 1 wherein each external receiver further comprises a processor operatively connected to memory for analyzing the electrocardiographic signal and storing episodes of electrocardiographic signals and further processing and verification.

12. The cardiac arrest monitor and alarm system of claim 1 wherein each external receiver comprises a GPS, activatable when the alarm is transmitted by the communication link to the remote transceiver.

13. The cardiac arrest monitor and alarm system of claim 1, wherein a location of a patient is identified using a least one of a triangulation calculation and a time-of-flight calculation.

14. The cardiac arrest monitor and alarm system of claim 1, wherein a location of a patient is identified using a triangulation calculation with cellular phone technology.

15. A cardiac arrest monitor and alarm system comprising:
a medical device having at least three electrodes positioned with respect to a patient's heart forming an orthogonal lead configuration monitoring an electrocardiographic signal of the patient's heart;
a microdevice operatively connected to the medical device, the microdevice analyzing the electrocardiographic signal and detecting a deviation from a normal heart electrical activity; and
a plurality of external receivers each electromagnetically connected to the microdevice, at least one external receiver detecting a signal from the microdevice and forwarding the signal to a telephone to activate a communication link with a remote transceiver, the microdevice comprising a plurality of sense amplifiers each receiving an electrocardiographic signal from one of an orthogonal lead and each sense amplifier emitting an amplified and digitized electrocardiographic signal to a microcontroller.

16. The cardiac arrest monitor and alarm system of claim 15, wherein the medical device is subcutaneous.

17. The cardiac arrest monitor and alarm system of claim 15 wherein the medical device is on a body surface of a patient.

18. The cardiac arrest monitor and alarm system of claim 15, wherein the microdevice is implantable.

19. The cardiac arrest monitor and alarm system of claim 15 wherein the telephone is programmed to automatically dial an emergency rescuer upon receiving the signal from at least one external receiver.

20. The cardiac arrest monitor and alarm system of claim 15 further comprising a short-wave radio operatively connected to each external receiver and transmitting the signal to the telephone.

21. The cardiac arrest monitor and alarm system of claim 15 further comprising a modulated carrier operatively connected to each external receiver and forwarding the signal to the telephone.

22. The cardiac arrest monitor and alarm system of claim 15 wherein each of the at least three electrodes is positioned on an exterior surface of the microdevice.

23. The cardiac arrest monitor and alarm system of claim 15 wherein the communication link transmits a heart electrocardiographic signal to the remote transceiver and provides a position identifier for locating a patient.

24. A method for detecting a deviation from a normal heart electrical activity and transmitting an alarm upon detecting the deviation, the method comprising:
monitoring an electrocardiographic signal of a heart organ using a medical device;
amplifying and digitizing the electrocardiographic signal;
executing a set of instructions to analyze the electrocardiographic signal;
detecting the deviation from the normal heart electrical activity and upon detecting the deviation, transmitting the alarm and the electrocardiographic signal to at least one of a plurality of external receivers electromagnetically connected to a microdevice operatively connected to the implantable medical device;
activating a programmed annunciator circuit to deliver the alarm and establish with a remote transceiver a communication link providing a communication between a person alerted by the alarm and the remote transceiver;
transmitting the electrocardiographic signal to the remote transceiver;
automatically providing an automatic location identification signal; and
activating a telephone to automatically dial a programmed emergency telephone number.

25. The method of claim 24 wherein the telephone is activated by transmitting a radio frequency signal from the microdevice to the telephone to automatically dial the programmed emergency telephone number.

26. The method of claim 24 wherein the telephone is activated by forwarding a signal from the microdevice to the telephone using a modulated carrier on a household power line.

27. The method of claim 24 wherein an analysis of the electrocardiographic signal comprises operating an algorithm for detecting ventricular activation and measurement of an interval between successive ventricular activations.

28. The method of claim 24 wherein an analysis of the electrocardiographic signal comprises operating an algorithm for detecting deviation from a baseline of a segment of the electrocardiographic signal following a QRS complex.

29. The method of claim 24 wherein analysis of the electrocardiographic signal comprises operating an algorithm for detecting a ST segment deviation.

30. The method of claim 24 wherein the deviation comprises one of an acute myocardial ischemia, a ventricular tachycardia and a ventricular fibrillation.

31. The method of claim 24 further comprising the step of instructing the person alerted by the alarm.

32. The method of claim 24 wherein the transmission of the alarm is delayed to disengage the alarm in a false detection of the deviation from the normal heart electrical activity.
